(19) United States
(12) Patent Application Publication
(10) Pub. No.: US 2021/0334566 A1
(43) Pub. Date: Oct. 28, 2021

(54) DETECTING A BIOMETRIC EVENT IN A NOISY SIGNAL

(71) Applicant: BIOBEATS GROUP LTD, London (GB)

(72) Inventors: Davide Morelli, Livorno (IT); David Plans, Woking (GB)

(73) Assignee: BIOBEATS GROUP LTD, London (GB)

(21) Appl. No.: 17/283,940

(22) PCT Filed: Oct. 8, 2019
(86) PCT No.: PCT/GB2019/052841

§ 371 Date: Apr. 8, 2021

(30) Foreign Application Priority Data
Oct. 8, 2018 (GB) 1816386.5

(51) Int. Cl. G06K 9/00; G06K 9/62

(52) U.S. Cl. CPC G06K 9/00885; G06K 2009/00939; G06K 9/6277; G06K 9/6247

(57) ABSTRACT

A method of detecting a biometric event in an input signal comprises: performing principal component analysis PCA on samples of a plurality of model signals to generate a transformation matrix having more informative components and less informative components, each model signal comprising a known signal which includes the biometric event to be detected; reducing a dimensionality of the transformation matrix by discarding one or more of the more informative components; transforming a plurality of samples of the input signal using the reduced dimensionality transformation matrix; determining a probability that the biometric event is present in the plurality of samples of the input signal, by calculating a predefined probability function for the transformed samples; and determining that the input signal includes the biometric event if the probability is higher than a threshold.

### TECHNICAL FIELD

The present invention relates to a method, apparatus and computer program for detecting a biometric event in a noisy signal using Principle Component Analysis (PCA). More particularly, but not exclusively, the present invention relates to detecting one or more heartbeats in an input signal.

### BACKGROUND

In many signal processing applications, it is necessary to determine whether or not a certain event is present in a noisy input signal. The presence of noise can cause errors in conventional threshold-based methods. For example, in the field of healthcare, a pulse rate is conventionally measured by counting heartbeats in an input signal such as a photoplethysmography (PPG) signal or electrocardiograph (ECG) signal, using a threshold-based method in which a heartbeat is counted when the signal crosses a certain threshold. Noise in the signal can result in errors, for example by causing the peak of an actual heartbeat to fall below the threshold, or by causing a spurious peak which exceeds the threshold and triggers the heartbeat detection algorithm when a heartbeat has not actually occurred. There is therefore a need in the art for an improved method of detecting biometric events such as heartbeats in noisy input signals.

The invention is made in this context.

### SUMMARY OF THE INVENTION

According to a first aspect of the present invention, there is provided a method of detecting a biometric event in an input signal, the method comprising: performing principal component analysis PCA on samples of a plurality of model signals to generate a transformation matrix having more informative components and less informative components, each of the model signals comprising a known signal which includes the biometric event to be detected; reducing a dimensionality of the transformation matrix by discarding one or more of the more informative components; transforming a plurality of samples of the input signal using the reduced dimensionality transformation matrix; determining a probability that the biometric event is present in the plurality of samples of the input signal, by calculating a predefined probability function for the transformed samples; and determining that the input signal includes the biometric event if the probability is higher than a threshold.

In some embodiments according to the first aspect, the plurality of samples of the input signal are selected by applying a time window to the input signal, the time window having the same duration as the plurality of model signals, and the method further comprises moving the window in time through the input signal and recalculating the probability function for each one of a plurality of positions of the window, to determine whether the biometric event is present at different times in the input signal.

In some embodiments according to the first aspect, the plurality of model signals are each arranged to have a peak amplitude at the same position within the signal, and in response to a determination that the input signal includes the biometric event the method further comprises identifying a time index of one of the plurality of samples of the input signal at an equivalent position to the position of the peak amplitude within the model signals, and recording the time index of the identified sample for the detected biometric event.

In some embodiments according to the first aspect, the biometric event comprises one of a heartbeat, a variation in a heartbeat and a user's activity. Where the biometric event comprises a heartbeat the plurality of model signals comprising a plurality of known heartbeat signals.

In some embodiments according to the first aspect in which the biometric event to be detected is a heartbeat, determining that the input signal includes a heartbeat comprises: identifying a probable heartbeat, in response to the probability being higher than the threshold; determining a time period between the probable heartbeat and an immediately preceding heartbeat in the input signal; and determining whether the probable heartbeat is an actual heartbeat based on a comparison between the determined time period and a known pulse rate.

In some embodiments according to the first aspect in which the biometric event to be detected is a heartbeat, determining whether the probable heartbeat is an actual heartbeat comprises: determining an expected interval between heartbeats based on the known pulse rate, and determining that the probable heartbeat is not an actual heartbeat if the determined time period differs by more than a threshold amount from the expected interval. For example, in some embodiments the threshold amount is ±30% of the expected interval.

In some embodiments the method of the first aspect comprises a further step prior to determining the probability, of setting the probability of the biometric event occurring to zero for a predefined time following each detection of a biometric event.

One way of reducing computational complexity and saving processing power is to set the probability to zero prior to performing the step of determining the probability during a time period where one know that any biometric event detected will not or is extremely unlikely to be the biometric event. In this regard a heartbeat for example has a maximum rate and therefore following detection of a heart beat there will be a short period of time where another heart beat will not occur. Setting the probability to zero for this period reduces the time period needing to be analysed and therefore saves processing power while also increasing accuracy, as false positives that may occur due to noise in this period are eliminated.

Alternatively, in some embodiments according to the first aspect in which the biometric event to be detected is a heartbeat, determining whether the probable heartbeat is an actual heartbeat comprises determining that the probable heartbeat is not an actual heartbeat if the determined time period is less than a predefined minimum time period. For example, in some embodiments the predefined minimum time period is 200 milliseconds. This is an alternative way of reducing the number of false positives by ruling out signals that occur in a period where one assesses a heartbeat cannot occur.

In some embodiments according to the first aspect in which the biometric event to be detected is a heartbeat, the method further comprises identifying a subject from which the input signal was obtained by comparing the transformation matrix to a plurality of stored transformation matrices, each associated with a particular subject.

In some embodiments according to the first aspect, the method further comprises validating the input signal by determining the standard deviation of the standard deviation of the input signal, wherein the input signal is rejected if the standard deviation of the standard deviation is higher than a preset threshold.

According to a second aspect of the present invention, there is provided a computer-readable storage medium arranged to store computer program instructions which, when executed, perform a method according to the first aspect.

According to a third aspect of the present invention, there is provided apparatus for detecting an biometric event in an input signal, the apparatus comprising a principal component analysis PCA unit configured to perform PCA on samples of a plurality of model signals to generate a transformation matrix having more informative components and less informative components, each of the model signals comprising a known signal which includes the biometric event to be detected, and to reduce a dimensionality of the transformation matrix by discarding one or more of the more informative components, a sample transformation unit configured to transform a plurality of samples of the input signal using the reduced dimensionality transformation matrix, a probability determining unit configured to determine a probability that the biometric event is present in the plurality of samples of the input signal, by calculating a predefined probability function for the transformed samples, and an biometric event detecting unit configured to determine that the input signal includes the biometric event if the probability is higher than a threshold.

According to a fourth aspect of the present invention, there is provided apparatus for detecting an biometric event in an input signal, the apparatus comprising a processing unit comprising one or more processors, and memory arranged to store computer program instructions which, when executed by the processing unit, cause the apparatus to: perform principal component analysis PCA on samples of a plurality of model signals to generate a transformation matrix having more informative components and less informative components; reduce a dimensionality of the transformation matrix by discarding one or more of the more informative components; transform a plurality of samples of the input signal using the reduced dimensionality transformation matrix; determine a probability that the biometric event is present in the plurality of samples of the input signal, by calculating a predefined probability function for the transformed samples; and determine that the input signal includes the biometric event if the probability is higher than a threshold.

In some embodiments according to the third or fourth aspect, the biometric event to be detected is a heartbeat and the plurality of model signals comprise a plurality of known heartbeat signals, and the apparatus further comprises a sensor configured to obtain the input signal by recording values of a physiological parameter over time. In some embodiments the sensor may be a photoplethysmography sensor.

### BRIEF DESCRIPTION OF THE DRAWINGS

Embodiments of the present invention will now be described, by way of example only, with reference to the accompanying drawings, in which:

FIG. 1 is a flowchart showing a method of determining whether a received signal sample includes a heartbeat, according to an embodiment of the present invention;

FIG. 2 illustrates a plurality of model heartbeats, according to an embodiment of the present invention;

FIG. 3 illustrates the plurality of model heartbeats of FIG. 2 after normalisation;

FIG. 4 illustrates the mean and standard deviation for each point in the normalised model heartbeats of FIG. 3;

FIG. 5 illustrates an example of an input signal containing two heartbeats, according to an embodiment of the present invention;

FIG. 6 illustrates a sliding probability function calculated for the signal of FIG. 5, according to an embodiment of the present invention;

FIG. 7 illustrates an example of an input signal containing two heartbeats with Gaussian noise at 0.3× the input signal power, according to an embodiment of the present invention;

FIG. 8 illustrates a sliding probability function calculated for the signal of FIG. 7, according to an embodiment of the present invention;

FIG. 9 illustrates an example of an input signal containing two heartbeats with Gaussian noise at 0.5× the input signal power, according to an embodiment of the present invention;

FIG. 10 illustrates a sliding probability function calculated for the signal of FIG. 9, according to an embodiment of the present invention;

FIG. 11 is a flowchart showing a method of determining whether a probable heartbeat is an actual heartbeat, according to an embodiment of the present invention;

FIG. 12 illustrates apparatus for determining whether a received signal sample includes a heartbeat, according to an embodiment of the present invention; and

FIG. 13 illustrates a series of graphs showing the second momentum of the input signal for a noisy PPG signal and for a clean PPG signal, according to an embodiment of the present invention.

### DETAILED DESCRIPTION

In the following detailed description, only certain exemplary embodiments of the present invention have been shown and described, simply by way of illustration. As those skilled in the art would realize, the described embodiments may be modified in various different ways, all without departing from the scope of the present invention. Accordingly, the drawings and description are to be regarded as illustrative in nature and not restrictive. Like reference numerals designate like elements throughout the specification.

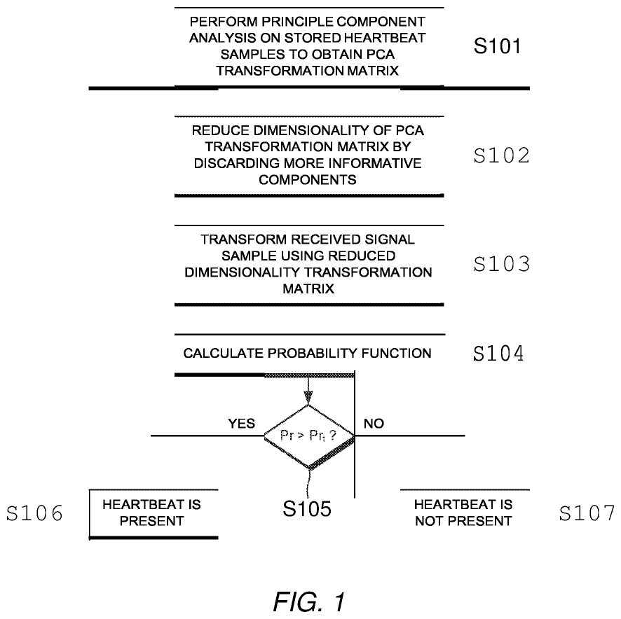

Referring now to FIG. 1, a flowchart showing a method of determining whether a received signal sample includes a heartbeat is illustrated, according to an embodiment of the present invention.

First, in step S101, principal component analysis (PCA) is performed on a plurality of heartbeat samples. Each one of the plurality of heartbeat samples can be referred to as a model heartbeat, and comprises a signal which is known to each contain a single heartbeat. For example, the model heartbeats can be extracted from a suitable biometric signal, such as a photoplethysmography (PPG) signal or electrocardiograph (ECG) signal, which contains heartbeats at known points in time within the signal. The model heartbeats can be recorded in advance and stored in suitable non-volatile computer-readable memory for analysis at a later time.

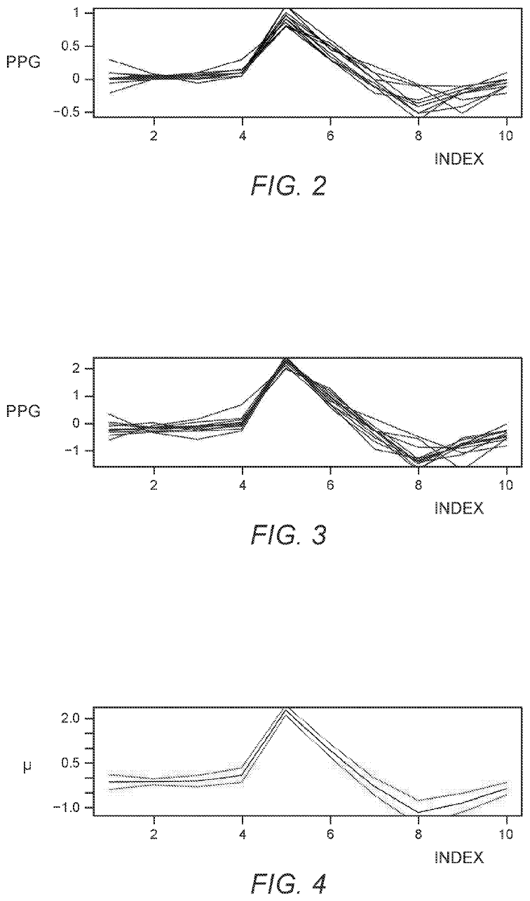

An example of a plurality of heartbeat samples is illustrated in FIG. 2, in which ii model heartbeats are plotted. In the present example each model heartbeat comprises ten samples at regular intervals in time, denoted by the sample index ion the x-axis. Although ii model heartbeats are illustrated in the present example, in other embodiments any number of model heartbeats may be provided.

The model heartbeats can be stored in an array with a number of rows i equal to the number of model heartbeats and a number of columns j equal to the number of samples in each model heartbeat. The element aᵢⱼ of the array therefore contains the j^{th} sample of the i^{th} model heartbeat. An example of an array containing ten samples for each of the eleven model heartbeats plotted in FIG. 2, in which each row contains the samples from one model heartbeat, is as follows:

$$\vert \begin{matrix}0 & 0 & 0.05 & 0.1 & 1 & 0.5 & 0 & - 0.5 & - 0.2 & - 0.1 \\ ( ) .01 & ( ) .05 & 0.07 & 0.15 & 0.8 & 0.55 & 0 & - 0.1 & - 0.5 & - 0.1 \\ - 0.05 & 0.0 & 0.01 & 0.05 & 1.1 & 0.45 & 0.2 & - 0.07 & - 0.3 & - 0.2 \\ 0.3 & 0.08 & 0 & 0.1 & 0.95 & 0.5 & 0.1 & - 0.09 & - 0.1 & - 0.0 \\ 0.02 & 0.07 & - 0.05 & 0.05 & 0.9 & 0.4 & - 0.1 & - 0.6 & - 0.15 & - 0.05 \\ - 0.2 & 0.01 & 0.1 & 0.3 & 0.85 & 0.3 & - 0.05 & - 0.5 & - 0.4 & - 0.1 \\ 0.1 & ( ) .03 & 0.02 & 0.1 & 0.79 & 0.5 & 0 & - 0.5 & - 0.2 & - 0.1 \\ 0.1 & 0.02 & 0.05 & 0.1 & 1.1 & 0.6 & 0.1 & - 0.4 & - 0.2 & - 0.05 \\ 0.01 & 0.03 & 0.05 & 0.1 & 0.96 & 0.35 & - 0.1 & - 0.4 & - 0.2 & 0 \\ 0.1 & 0.05 & 0.07 & 0.09 & 0.9 & 0.3 & - 0.11 & - 0.35 & - 0.15 & 0.1 \\ 0 & 0.04 & 0.1 & 0.14 & 0.8 & 0.3 & - 0.2 & - 0.3 & - 0.1 & - 0.01\end{matrix} \vert$$

FIG. 3 illustrates the model heartbeats from FIG. 2 after z-normalising each model heartbeat. The normalisation process can involve centring and/or scaling the model heartbeats. Normalising the model heartbeats in this way allows signals with different ranges of amplitude values to be compared to one another, and may be applied, for example, when the absolute values of the amplitude vary significantly from one model heartbeat to the next. In other embodiments the normalisation step may be omitted, for example when the range of amplitude values within each model heartbeat is the same or similar among the plurality of model values.

FIG. 4 is a graph plotting the mean and standard deviation for each sample index in the normalised model heartbeats of FIG. 3. As shown in FIG. 4, the standard deviation of the model heartbeat values can be quite different at different points within the heartbeat. In the present example, the model heartbeat values at time index i=8 have a standard deviation of approximately 0.5, whereas the model heartbeat values at time index i=2 have a standard deviation of approximately 0.1. The points with smaller standard deviations are more indicative of whether a particular sample can be considered as belonging to the distribution of model heartbeat values. For instance, in the example shown in FIG. 4, a point with an amplitude value that lies 0.5 away from the mean value of the distribution at index i=2 is unlikely to be part of the distribution, and therefore is unlikely to be part of a heartbeat, since the standard deviation at this index is 0.1. In contrast, a point with an amplitude value that lies the same distance away from the mean (μ±0.5) at index i=8 could potentially be part of the distribution, since the standard deviation at this index is 0.5. Therefore, in embodiments of the present invention, more importance can be given to dimensions (array indexes) which have lower standard deviation when determining whether or not an input signal contains a heartbeat.

The indexes in an array of model heartbeat samples taken from PPG measures are not independent, and so the covariance matrix will not be diagonal. However, they can be transformed into a space in which dimensions are orthogonal using PCA. In the present embodiment, in step S101 PCA is performed on the plurality of model heartbeats to generate a transformation matrix. The elements of the PCA transformation matrix are ordered according to variance, with the first element having the most variance and the last element having the least variance. The elements with higher variance can be referred to as more informative components, and the elements with lower variance can be referred to as less informative components. That is, the less informative components have lower variances than the more informative components.

It is known to perform dimensionality reduction on a PCA matrix by retaining the components with most variance and discarding the components with less variance, on the basis that the components with most variance contain more information about the differences between the signals that were used to calculate the PCA matrix. In contrast however, in embodiments of the present invention the dimensionality is reduced by discarding the more informative components, that is, the components of the PCA matrix which have higher variances. As explained above, the inventors of the present invention have noted that the components with less variance (i.e., the less informative components) give a better indication of whether a particular signal belongs to the distribution than components with more variance, since points lying far from the mean values on dimensions which have less variance will indicate that the sample does not belong to the distribution.

Therefore in the present embodiment, in step S102 the dimensionality of the PCA transformation matrix is reduced from n to k by discarding the (n−k) most informative components, where n is the size of the original PCA transformation matrix, and k is the number of retained components. This is equivalent to transforming the samples of the model heartbeats from the original PPG space into a space where the dimensions are orthogonal, ensuring that only those points with small variance are retained. For example, by performing dimensionality reduction the number of dimensions in the transformation matrix may be reduced from 100 to 10. In some embodiments, dimensionality reduction can be performed by discarding a fixed number (n-k) of the more informative components. In other embodiments, dimensionality reduction can be performed by discarding any components with a variance higher than a certain threshold.

Next, in step S103 the reduced-dimensionality transformation matrix is applied to samples of the input signal. This has the effect of transforming the input signal into a space in which dimensions are orthogonal, and where the dimensions are ordered by the amount of variance. In embodiments in which the model heartbeats were normalised before performing PCA, the same transformation in terms of centering and/or scaling the amplitude values may also be applied to the samples of the input signal, before applying a rotation using the reduced-dimensionality transformation matrix.

Then, in step S104 a predefined probability function is calculated for the transformed samples. The probability function calculates the probability that the samples of the input signal belong to the distribution that was used to create the transformation matrix, specifically, the distribution of sample values for a plurality of model heartbeats. The output of the probability function is therefore related to the probability that the input signal includes a heartbeat.

Next, in step S105 the probability that was calculated in step S104 is compared against a threshold. If the probability is higher than the threshold, it is determined that the input signal contains a heartbeat. On the other hand, if the probability is lower than the threshold, it is determined that the input signal does not contain a heartbeat.

By reducing the dimensionality of the PCA transformation matrix, as described above in relation to step S102 of FIG. 1, the computational burden can be reduced since fewer calculations must be performed. Also, retaining the less informative components ensures that heartbeats can still be reliably detected despite the reduction in size of the PCA transformation matrix. Embodiments of the present invention can therefore provide an accurate, computationally efficient method of determining whether an input signal contains a heartbeat. Without dimensionality reduction, potentially a very high number of calculations would need to be performed during every sample period.

For example, if a smartphone sensor is used to record a PPG signal with a total duration of 1 second at a sampling rate of 240 Hz, the full covariance matrix would have a size of 240×240. Without dimensionality reduction, this would result in 240×240=57,600 multiplications having to be performed 240 times each second. By comparison, if dimensionality reduction is performed by retaining the 10 least informative components and discarding the remaining more informative components, then it is only necessary to perform 240×10=2,400 multiplications during each sampling period, followed by calculating the disjoint probability of 10 points, an operation which is O(n) and therefore involves a number of operations in the same order of magnitude as the number of reduced dimensions. Therefore embodiments of the present invention may be particularly advantageous in applications where the available processing resources are limited, for example in wearable devices or other types of mobile device such as smartphones.

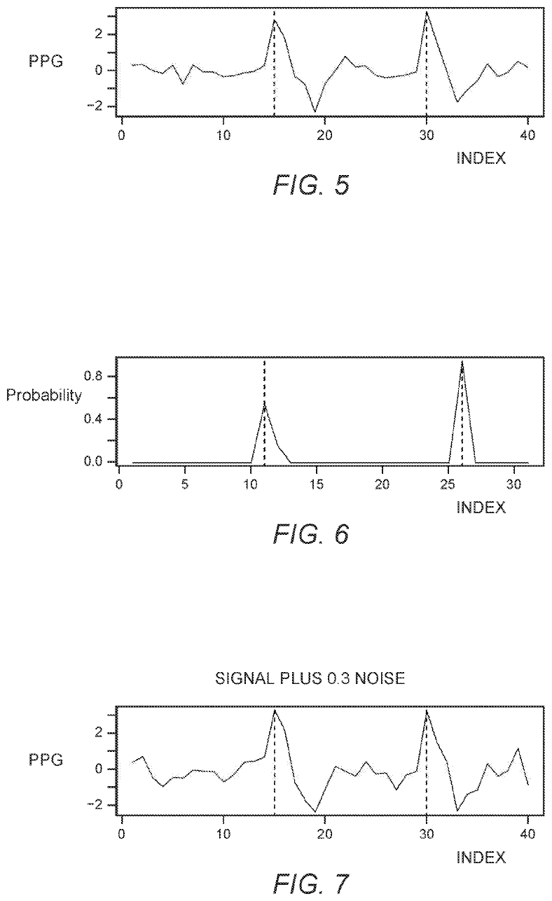

Referring now to FIG. 5, an example of an input signal containing two heartbeats is illustrated, according to an embodiment of the present invention. In FIG. 5 the PPG amplitude is plotted against the sample index. In the present embodiment the input signal comprises forty samples in total, numbered from 1 to 40. In order to determine whether a heartbeat is present at a certain point in the input signal, a plurality of samples can be selected within a time window that is equal in width (i.e., duration) to the length of the model heartbeats that were used to obtain the PCA transformation matrix. The selected samples are then processed using a method such as the one shown in FIG. 1 in order to determine a probability that a heartbeat is present in the part of the input signal which falls within the time window.

In some embodiments, a sliding probability function can be calculated by moving the window in time through the input signal and recalculating the probability function for each one of a plurality of positions of the window, to determine whether a heartbeat is present at different times in the input signal. An example of a sliding probability function calculated for the signal of FIG. 5 is shown in FIG. 6.

In the example shown in FIG. 6, a probability value is calculated for each position of the time window using the reduced-dimensionality transformation matrix derived from the normalised model heartbeats shown in FIG. 3, in which each model heartbeat comprises ten samples. Therefore in the present embodiment, the width of the time window is set to 9×S, where S is the sampling rate of the input signal, such that the time window encompasses ten samples of the input signal. In another embodiment the model heartbeats may comprise a different number of samples, and the width of the time window may be adjusted accordingly.

In the present embodiment the model heartbeats are arranged so as to have the amplitude peak at sample index j=5, which occurs four sampling intervals after the first sample. The probability function calculated at step S104 of FIG. 1 will have a maximum when the peak amplitude of a heartbeat within the input signal is located at an equivalent position in the time window to the position of the peak amplitude in the model heartbeats. Therefore in the present embodiment, the probability function will have a maximum when the window is positioned so that the peak amplitude of a heartbeat in the input signals lies four sampling intervals after the start of the window.

In FIG. 6, the value of the sliding probability function is plotted against the index of the sample at the start of the window. As shown in FIG. 6, the probability function includes two peaks, showing that the input signal contains two heartbeats. The first peak in the probability function occurs at an index of ii, from which it can be determined that the peak amplitude of the first heartbeat occurs at a time index of 11+4=15, as shown in FIG. 5. The second peak in the probability function occurs at an index of 26, from which it can be determined that the peak amplitude of the first heartbeat occurs at a time index of 26+4=30, as shown in FIG. 5. In response to a peak being detected in the sliding probability function, the time index of the sample at an equivalent position within the time window to the position of the peak within the known heartbeat signal can be identified and used to record the position of the detected heartbeat.

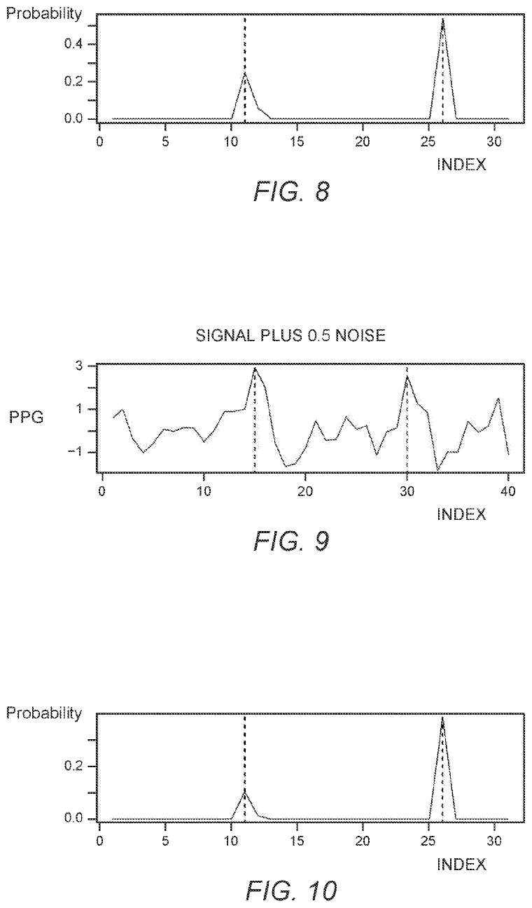

Embodiments of the present invention can also reliably detect heartbeats in noisy input signals. FIGS. 7 and 8 illustrate an input signal and sliding probability function, respectively, for an example in which the input signal contains Gaussian noise with a noise power level equal to 30% of the input signal power. FIGS. 9 and 10 illustrate an input signal and sliding probability function, respectively, for an example in which the input signal contains Gaussian noise with a noise power level equal to 50% of the input signal power. The input signals in FIGS. 7 and 9 are based on the input signal of FIG. 5, with added Gaussian noise. As shown in FIGS. 8 and 10, even with relatively high noise levels a peak is still clearly visible in the sliding probability function for each of the two heartbeats.

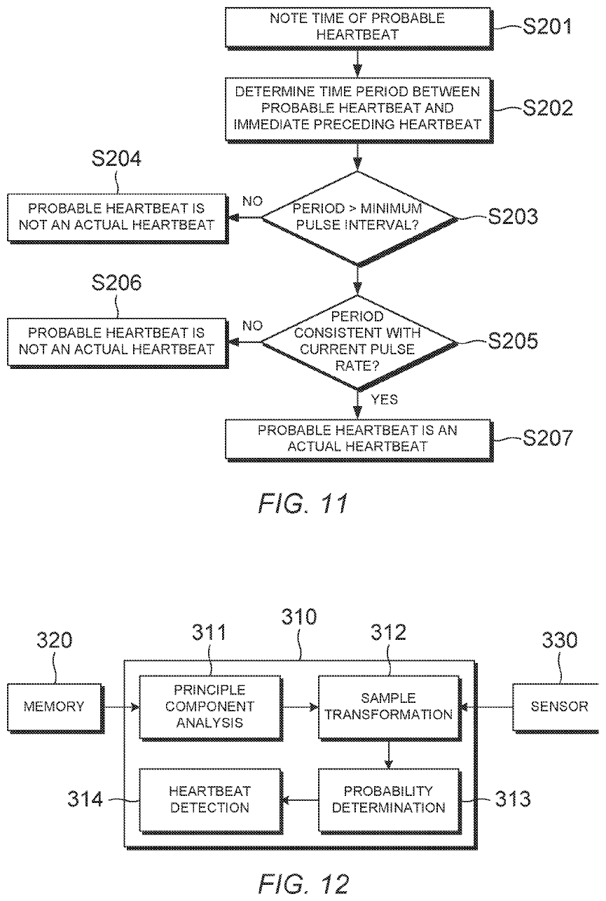

Referring now to FIG. 1i, a flowchart is illustrated showing a method of determining whether a probable heartbeat is an actual heartbeat, according to an embodiment of the present invention. The steps shown in FIG. 11 can be carried out during step S106 of the method shown in FIG. 1 once a probable heartbeat has been detected at step S105.

First, in step S201 the time at which the probable heartbeat occurs in the input signal is noted. For example, when a sliding probability function is used as described above, the time of the probable heartbeat can be determined based on the current starting point of the time window and the known position of the heartbeat in the model heartbeats.

Next, in step S202 the time period between the probable heartbeat and the immediately preceding heartbeat in the input signal is determined. If the probable heartbeat is an actual heartbeat, then this time period represents the interval between consecutive heartbeats. In step S203, it is checked whether the determined period is greater than a predefined minimum time period, which can be referred to as a minimum pulse interval. The minimum pulse interval may be set to be lower than the shortest interval that would be expected for a realistic maximum heart rate. If the determined time period is found to be less than the minimum pulse interval, then in step S204 it is determined that the probable heartbeat cannot be an actual heartbeat.

In the present embodiment the minimum pulse interval is set to 200 ms (milliseconds), which is equivalent to a heart rate of (1000/200)*60=300 bpm (beats per minute). Since the maximum heart rate of a human is generally expected to be around 200-220 beats per minute, it can be assumed that if the time period calculated in step S202 is less than 200 ms, the probable heartbeat cannot be an actual heartbeat since the pulse rate could not be that high. It will be understood that in other embodiments a different minimum pulse interval may be set. For example, in some embodiments a value of less than or equal to 270 ms may be used, equivalent to a heart rate of approximately 220 bpm.

If the determined time period is found to be greater than the minimum pulse interval, then the probable heartbeat may be an actual heartbeat. Accordingly, in step S205 the time period that was determined in step S203 is compared to an interval between consecutive heartbeats that would be expected based on a current pulse rate. For example, the current pulse rate can be determined based on the total number of heartbeats that have been detected within a preceding predefined time period or can be determined based on the average interval between a predefined number of heartbeats.

In step S205, the time period is determined to be consistent with the expected interval if it differs from the expected interval by less than a threshold amount. If the time period is not found to be consistent with the expected interval, then in step S206 it is determined that the probable heartbeat cannot be an actual heartbeat. On the other hand, if the time period is consistent with the expected interval, then in step S207 it is determined that the probable heartbeat is an actual heartbeat.

In step S205, the threshold for determining whether or not the time period is consistent with the expected interval can be defined in relative or absolute terms, for example as a percentage of the expected interval or as a fixed time difference. In the present embodiment the time period determined in step S202 is deemed to be consistent with the expected interval if it is within ±30% of the expected interval. However, in other embodiments a different threshold may be used.

The checks provided in steps S203 and S205 may be applied in order to verify whether or not a probable heartbeat detected using a method such as the one shown in FIG. 1 is an actual heartbeat. In some embodiments, the tests shown in steps S203 and S205 may be performed in a reverse order, or one of the tests may be omitted. Furthermore, in some embodiments both tests may be omitted, and a heartbeat can be recorded whenever the probability exceeds the threshold in step S105.

In some embodiments a similar logic may be applied before using a process such as the one shown in FIG. 1 to calculate a probability that a heartbeat is present. For example, in some embodiments when a sliding probability function is used, the probability can be set to be zero for a certain time after a heartbeat has been detected, equal to the minimum pulse interval. Since the probability is automatically set to zero during this period, it is not necessary to calculate the probability function for positions of the time window during this period, and therefore the computational burden can be reduced. Similarly, when a heartbeat is detected, the expected time at which the next heartbeat should occur can be determined based on the current pulse rate. To reduce the computational burden even further, the sliding probability function may only be calculated within a certain range of the expected time of the next heartbeat, for example within a range equivalent to ±30% of the expected interval between consecutive heartbeats. Outside of this range, the probability can be automatically set to zero without having to calculate the probability function using a method such as the one in FIG. 1.

Referring now to FIG. 12, apparatus for determining whether a received signal sample includes a heartbeat is schematically illustrated, according to an embodiment of the present invention. The apparatus includes a processing unit 310, memory 320 in the form of a suitable computer-readable storage medium, and a sensor 330. The sensor 330 is configured to provide the input signal to the processing unit 310, by recording values of a physiological parameter over time. For example, the sensor 330 may be a PPG sensor or may be any other type of sensor capable of recording a signal in which a heartbeat may be detected.

Depending on the embodiment, the processing unit 310, memory 320 and sensor 330 may be embodied in the same physical device or may be physically separate from one another. For example, the processing unit and memory may be included in one device, such as a smartphone, and the sensor 330 may be included in a physically separate device that can communicate with the processing unit 310 via a suitable wired or wireless connection, for example in a wearable device such as a smartwatch which includes an integrated PPG sensor, or a chest strap with integrated heart rate sensor.

As shown in FIG. 12, in the present embodiment the processing unit 310 comprises a PCA unit 311, a sample transformation unit 312, a probability determining unit 313, and a heartbeat detecting unit 314. Depending on the embodiment, the different elements of the processing unit 310 may be embodied as separate hardware elements or as software modules. When a software implementation is used, the memory 320 may be used to store computer program instructions which implement the functions of the PCA unit 311, sample transformation unit 312, probability determining unit 313, and heartbeat detecting unit 314 when executed by one or more processors in the processing unit 310.

The PCA unit 311 is configured to perform PCA on samples of a plurality of known heartbeat signals to generate a transformation matrix, and to reduce a dimensionality of the transformation matrix by discarding one or more of the more informative components, as described above in relation to steps S101 and S102 of FIG. 1. The sample transformation unit 312 is configured to transform a plurality of samples of the input signal using the reduced dimensionality transformation matrix, as described above in relation to step S103 of FIG. 1. The probability determining unit 313 is configured to determine a probability that the input signal includes a heartbeat, by calculating a predefined probability function for the transformed samples, as described above with reference to step S104 of FIG. 1. Finally, the heartbeat detecting unit 314 is configured to determine that the input signal includes a heartbeat based on the probability calculated by the probability determining unit 313. In some embodiments the heartbeat detecting unit 314 may also carry out additional checks such as those described above with reference to FIG. 1i, to verify whether the probable heartbeat is an actual heartbeat.

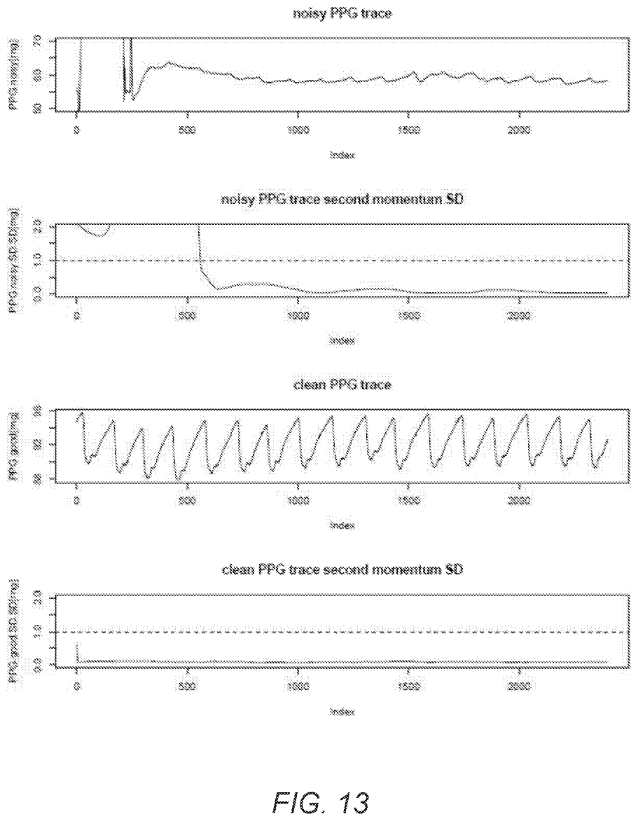

Embodiments of the present invention have been described which can be used to determine whether an input signal contains a heartbeat. In some embodiments, the input signal can be validated before proceeding to check whether a heartbeat is present, to avoid unnecessarily expending processing resources when the input signal is unsuitable for detecting a heartbeat. For example, in one embodiment the input signal can be validated by determining the standard deviation of the standard deviation of the input signal, which may also be referred to as the second momentum of the input signal. FIG. 13 illustrates a series of graphs showing the second momentum of the input signal for a noisy PPG signal and for a clean PPG signal. When the second momentum of the input signal is higher than a threshold, as shown in the second graph from the top in FIG. 13, the input signal can be rejected on the basis that the signal is too noisy to allow a heartbeat to be reliably detected. On the other hand, the input signal can be accepted if the second momentum is lower than the threshold, as shown in the bottom graph in FIG. 13, and the system may continue to process the signal using methods as described above, in order to detect heartbeats in the signal.

Finally, embodiments of the present invention have been described in which a PCA transformation matrix is derived from a plurality of model heartbeats. In some embodiments, the system can adapt to a particular individual's characteristics by updating the model heartbeats using heartbeats extracted from the input signal. This can improve the accuracy for that particular individual, by training the system to recognize the characteristic waveform of that user's heartbeat. Furthermore, in some embodiments a plurality of PCA transformation matrices may be stored for different users, enabling the system to identify a subject from which the input signal was obtained by comparing the transformation matrix to the plurality of stored transformation matrices.

Embodiments of the present invention have been described in relation to detecting heartbeats in physiological signals such as PPG or ECG signals. However, in other embodiments of the invention the same principles disclosed above may be applied to process different types of biometric signals. In general, the PCA-based techniques disclosed herein can be used to detect any type of biometric event in a noisy signal. For example, in some embodiments the PCA-based event detection method may be applied to detect a user performing a certain activity such as a step from a noisy signal indicative of a user's movement.

Whilst certain embodiments of the invention have been described herein with reference to the drawings, it will be understood that many variations and modifications will be possible without departing from the scope of the invention as defined in the accompanying claims.

A person of skill in the art would readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The embodiments are also intended to cover computers programmed to perform said steps of the above-described methods.

Features described in the preceding description may be used in combinations other than the combinations explicitly described.

1. A method of detecting a biometric event in an input signal, the method comprising:
performing principal component analysis PCA on samples of a plurality of model signals to generate a transformation matrix having more informative components and less informative components, each of the model signals comprising a known signal which includes the event to be detected;
reducing a dimensionality of the transformation matrix by discarding one or more of the more informative components;
transforming a plurality of samples of the input signal using the reduced dimensionality transformation matrix;
determining a probability that the event is present in the plurality of samples of the input signal, by calculating a predefined probability function for the transformed samples; and
determining that the input signal includes the event if the probability is higher than a threshold.

2. The method of claim 1, wherein the plurality of samples of the input signal are selected by applying a time window to the input signal, the time window having the same duration as the plurality of model signals, the method further comprising:
moving the window in time through the input signal and recalculating the probability function for each one of a plurality of positions of the window, to determine whether the event is present at different times in the input signal.

3. The method of claim 1, wherein the plurality of model signals are each arranged to have a peak amplitude at the same position within the signal, and in response to a determination that the input signal includes the event the method further comprises:
identifying a time index of one of the plurality of samples of the input signal at an equivalent position to the position of the peak amplitude within the model signals; and
recording the time index of the identified sample for the detected event.

4. The method of claim 1, wherein the biometric event comprises one of a heartbeat, a variation in a heartbeat and a user's activity.

5. The method of claim 1, wherein the biometric event to be detected is a heartbeat, and the plurality of model signals comprise a plurality of known heartbeat signals.

6. The method of claim 1, wherein determining that the input signal includes a heartbeat comprises:
identifying a probable heartbeat, in response to the probability being higher than the threshold;
determining a time period between the probable heartbeat and an immediately preceding heartbeat in the input signal; and
determining whether the probable heartbeat is an actual heartbeat based on a comparison between the determined time period and a known pulse rate.

7. The method of claim 6, wherein determining whether the probable heartbeat is an actual heartbeat comprises:
determining an expected interval between heartbeats based on the known pulse rate, and
determining that the probable heartbeat is not an actual heartbeat if the determined time period differs by more than a threshold amount from the expected interval.

8. The method of claim 6 or 7, wherein the threshold amount is ±30% of the expected interval.

9. The method of claim 1, comprising a further step prior to determining the probability, of setting the probability of the biometric event occurring to zero for a predefined time following each detection of a biometric event.

10. The method of claim 6, wherein determining whether the probable heartbeat is an actual heartbeat comprises:
determining that the probable heartbeat is not an actual heartbeat if the determined time period is less than a predefined time period.

11. The method of claim 9, wherein the predefined time period is set to less than or equal to 200 milliseconds.

12. The method of claim 1, further comprising:
identifying a subject from which the input signal was obtained by comparing the transformation matrix to a plurality of stored transformation matrices, each associated with a particular subject.

13. The method of claim 1, further comprising:
validating the input signal by determining the standard deviation of the standard deviation of the input signal,
wherein the input signal is rejected if the standard deviation of the standard deviation is higher than a preset threshold.

14. (canceled)

15. Apparatus for detecting a biometric event in an input signal, the apparatus comprising:
a principal component analysis PCA unit configured to perform PCA on samples of a plurality of model signals to generate a transformation matrix having more informative components and less informative components, each of the model signals comprising a known signal which includes the biometric event to be detected, and to reduce a dimensionality of the transformation matrix by discarding one or more of the more informative components;
a sample transformation unit configured to transform a plurality of samples of the input signal using the reduced dimensionality transformation matrix;
a probability determining unit configured to determine a probability that the biometric event is present in the plurality of samples of the input signal, by calculating a predefined probability function for the transformed samples; and
a biometric event detecting unit configured to determine that the input signal includes the biometric event if the probability is higher than a threshold.

16. Apparatus for detecting a biometric event in an input signal, the apparatus comprising:
a processing unit comprising one or more processors; and
memory arranged to store computer program instructions which, when executed by the processing unit, cause the apparatus to:
perform principal component analysis PCA on samples of a plurality of model signals to generate a transformation matrix having more informative components and less informative components, each of the model signals comprising a known signal which includes the biometric event to be detected;
reduce a dimensionality of the transformation matrix by discarding one or more of the more informative components;
transform a plurality of samples of the input signal using the reduced dimensionality transformation matrix;
determine a probability that the biometric event is present in the plurality of samples of the input signal, by calculating a predefined probability function for the transformed samples; and
determining that the input signal includes the biometric event if the probability is higher than a threshold.

17. The apparatus of claim 15, wherein the biometric event to be detected is a heartbeat and the plurality of model signals comprise a plurality of known heartbeat signals, the apparatus further comprising:
a sensor configured to obtain the input signal by recording values of a physiological parameter over time.

18. The apparatus of claim 17, wherein the sensor is a photoplethysmography sensor.

19. The apparatus of claim 16, wherein the biometric event to be detected is a heartbeat and the plurality of model signals comprise a plurality of known heartbeat signals, the apparatus further comprising:
a sensor configured to obtain the input signal by recording values of a physiological parameter over time.
